(19) United States
(12) Patent Application Publication
(10) Pub. No.: US 2020/0131558 A1
(43) Pub. Date: Apr. 30, 2020

(54) PAPER-BASED ASSAY FOR ANTIMICROBIAL RESISTANCE

(71) Applicant: Colorado State University Research Foundation, Fort Collins, CO (US)

(72) Inventors: Charles S. HENRY, Fort Collins, CO (US); Katherine BOEHLE, Fort Collins, CO (US); Brian J. GEISS, Fort Collins, CO (US)

(73) Assignee: Colorado State University Research Foundation, Fort Collins, CO (US)

(21) Appl. No.: 16/606,705

(22) PCT Filed: Apr. 20, 2018
(86) PCT No.: PCT/US18/28534

§ 371 Date: Oct. 18, 2019

Related U.S. Application Data
(60) Provisional application No. 62/488,288, filed on Apr. 21, 2017.

(51) Int. Cl. C12Q 1/34; G01N 31/22; C12Q 1/04; G01N 1/28

(52) U.S. Cl. CPC C12Q 1/34; G01N 1/28; C12Q 1/04; G01N 31/22

(57) ABSTRACT

Antimicrobial resistance (AMR), the ability of a bacterial species to resist the action of an antimicrobial drug, has been on the rise due to the widespread use of antimicrobial agents, and one of the many ways AMR can spread is through contaminated water sources. To monitor these water sources, we have developed an inexpensive, fast assay using a paper-based analytical device (PAD) that can test for the presence of β-lactamase-mediated resistance as one major form of AMR that has reliably detected resistance in sewage water.

### RELATED APPLICATIONS

This application claims priority under 35 U.S.C. § 119(e) to U.S. Provisional Patent Application No. 62/488,288, filed Apr. 21, 2017, which is incorporated herein by reference.

### GOVERNMENT SUPPORT

This invention was made with government support under Grant No. 16-7400-0589-CA awarded by the USDA. The government has certain rights in the invention.

### BACKGROUND OF THE INVENTION

The introduction of antimicrobial agents in the early 20^{th} century revolutionized medicine, significantly decreasing morbidity and mortality. However, due to the widespread use of antimicrobial agents and the genetic plasticity of bacteria, more pathogens have developed the ability to resist these drugs, giving rise to antimicrobial resistant (AMR) bacteria. According to the World Health Organization (WHO), AMR costs approximately $21 B to $34 B annually within the United States alone, and is predicted to surpass heart disease as the number one cause of death worldwide by 2050. Contaminated water is a significant source of infection and outlet for the spread of AMR bacteria. AMR propagation in water is further advanced through contamination by antimicrobial agents, which results in the selective proliferation of AMR bacteria, and the horizontal gene transfer of resistance from AMR bacteria to non-AMR bacteria. Due to its significant role, many bodies of water have been studied for the presence of AMR bacteria including urban wastewater, irrigation water, and drinking water in China to name a few.

Growth inhibition assays, the assessment of bacterial growth in the presence of antimicrobial agents, is the gold standard for detecting AMR bacteria. While growth inhibition assays provide reliable results, they also require samples to be sent to a central laboratory to complete testing. In addition to transportation time, these methods require at least overnight (12-16 hr) incubation, trained laboratory personnel to execute the procedure and analyze results, and expensive instrumentation. Alternative methods for detecting AMR bacteria have also been developed, including expanded microarrays, microfluidic devices fabricated with poly-dimethysiloxane (PDMS), and paper-based culture devices. While these are all promising systems, they also require expensive equipment, long times, or trained personnel.

Paper-based analytical devices (PADs) have shown significant promise as an alternative platform for performing diagnostics. PADs have been developed for a variety of applications, including point-of-care (POC) diagnostics and environmental monitoring. Because of AMR concerns in both developed and developing countries, the WHO specifically mentions in their Global Action Plan for Antimicrobial Resistance the need for portable and inexpensive diagnostic tools.

Accordingly, a rapid, disposable, and inexpensive device that does not require instrumentation or trained laboratory personnel for analysis is still needed to monitor AMR bacteria in the field and diagnose AMR infections at the point-of-care.

### SUMMARY

PADs offer a cost effective platform because the starting substrate materials are inexpensive (often less than $0.01US), the manufacturing techniques are well established, and the reagents (the most expensive part) are deposited in small amounts (μg-pg). Many diagnostic motifs exist for PADs, but few have detected naturally-produced enzymes. Our group reported colorimetric and electrochemical assays to detect bacteria from food and water sources using the enzymes they produce. This same detection motif can be used for detecting AMR, as some antimicrobial properties can be traced back to enzymes responsible for deactivating antibiotics.

Accordingly, this disclosure provides a system for beta-lactamase enzyme detection comprising:
a) a planar cellulose-based mesh comprising a first surface having a hydrophobic perimeter, a hydrophobic surface opposite the first surface, and a chromogenic indicator dispersed in the mesh within the hydrophobic perimeter; and
b) a portable digital imaging device that records color images;

wherein the imaging device records a color image of the chromogenic indicator, wherein a beta-lactamase enzyme is detected by a change in the color of the chromogenic indicator when in contact with a beta-lactamase enzyme.

This disclosure also provides a method of detecting antimicrobial resistant (AMR) bacteria with the system disclosed above, comprising:
a) contacting a water sample with the chromogenic indicator dispersed in the mesh within the hydrophobic perimeter to form a mixture in the mesh;
b) incubating the mixture;
c) recording the color of the chromogenic indicator; and
d) analyzing the chromogenic indicator for a color change;

wherein a beta-lactamase enzyme from AMR bacteria that expresses the beta-lactamase enzyme is detected in the water sample by the change in the color of the chromogenic indicator relative to a control sample within a blank hydrophobic perimeter when the chromogenic indicator is contacted by the beta-lactamase enzyme.

Additionally, this disclosure provides a method for detecting beta-lactamase enzyme comprising:
a) drying one or more aliquots of a nitrocefin indicator on a sheet of absorbent paper comprising a first surface, one or more hydrophobic perimeters at the first surface, and a hydrophobic surface opposite the first surface, wherein a dried aliquot of the nitrocefin indicator is dispersed in the paper within the hydrophobic perimeter;
b) contacting a sample with the dried aliquot of the nitrocefin indicator to form a mixture in the paper; and
c) incubating the mixture;

wherein a beta-lactamase enzyme in a sample comprising the beta-lactamase enzyme is detected by the change in the color of the nitrocefin indicator relative to a control sample when the nitrocefin indicator contacts the beta-lactamase enzyme.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Reaction overview of β-lactamase and nitrocefin. Hydrolysis of the β-lactam ring in nitrocefin, mediated by β-lactamase, results in a distinct color change from yellow to red, making a visually detectable and user-friendly test.

FIG. 2. Optimization of β-lactam-resistant bacteria detection. (A) The paper-based tests were used for serial dilutions of bacteria that were both positive and negative for expressing β-lactamase to demonstrate specificity. (B) β-lactamase expressing bacteria was mixed with either non-β-lactamase expressing bacteria or pure media to determine if non-resistant bacteria would interfere with the reaction. (C) To determine if bacteria lysis would result in more sensitive detection, the reaction rate of sonicated bacteria was compared to intact bacteria. Error bars denote s.d. where n=3.

FIG. 3. Comparing nitrocefin detection methods. (A) Detecting color change using UV-vis spectrophotometry in a plate reader yielded the same limit-of-detection of 10⁶ CFU/mL as observed on paper. (B) Drying nitrocefin on paper before adding sample yielded similar or slightly more sensitive results compared to adding nitrocefin solution to the bacteria sample on paper. Error bars denote s.d. where n=3.

FIG. 4. Detecting β-lactam resistance in urban sewage water. Samples of influent and effluent water were obtained and incubated in media for 12 hr. Samples were obtained every 2 hr for testing and both the influent and effluent tested positive for β-lactam resistance, which was confirmed by traditional culture methods. Error bars denote s.d. where n=3.

FIG. 5. Detecting β-lactam resistance in bacterial isolates. Different bacteria species were isolated from environmental samples and tested for individual resistance using the paper-based test. There have been no false positives, and one false negative (Chromobacterium violaceum isolated from the influent of urban sewage water).

FIG. 6. Device fabrication and Data Analysis. (A) Devices were developed by printing wax on Whatman chromatography grade 4 paper, then heated on a hot plate to melt the wax through the pores, creating a defined hydrophobic barrier. The back of the device sheet was then covered in packing tape to prevent sample leakage. (B) Devices were imaged using a cardboard box lined with copy paper and a hole on the top that allows for a camera to view and image the devices. These images were then wirelessly sent to a computer to analyze using ImageJ software.

FIG. 7. Nitrocefin and β-lactamase reaction optimization on paper. (A) β-lactamase enzyme was reacted with nitrocefin using different pH buffers to determine the optimal reaction pH where pH 7.5 was selected. (B) Optimal nitrocefin concentration was determined using change in signal from starting color intensity of nitrocefin alone (before reaction) and increase in color intensity (after reaction). Nitrocefin concentrations above 1 mM would be too dark before adding sample to distinguish between positive and negative samples, therefore a lower concentration of 0.5 mM was selected. (C) Different concentrations of β-lactamase enzyme were reacted with 0.5 mM nitrocefin to determine the lowest concentration of β-lactamase that could be detected before moving onto live bacteria. The enzyme limit-of-detection was determined to be around 10 mU/mL. (D) Optimal nitrocefin concentration to dry in paper was determined using change in signal similar to nitrocefin in solution. (E) The paper-based devices were used to determine kinetic values by reacting 1 U/mL of β-lactamase with nitrocefin between 0.1 and 0.7 mM. Error bars denote s.d. where n=3 for all graphs.

FIG. 8. Comparing the PAD test to an ESBL-selecting plate, antibiotic susceptibility testing, and PCR gene analysis. According to ESBL-selecting plates, there were two false positives (#7 and #20). However, when compared to antibiotic susceptibility testing and PCR, these bacterial isolates were resistant to at least two penicillin antibiotics and had an ESBL gene in their genome.

### DETAILED DESCRIPTION

Antimicrobial resistance (AMR), the ability of a bacterial species to resist the action of an antimicrobial drug, has been on the rise due to the widespread use of antimicrobial agents. Per the World Health Organization, AMR has an estimated annual cost of $34 B in the US, and is predicted to be the number one cause of death worldwide by 2050. One way AMR bacteria can spread, and where individuals can contract AMR infections, is through contaminated water. Monitoring environment AMR bacteria currently requires samples be transported to a central laboratory for slow and labor intensive tests. We have developed an inexpensive assay using paper-based analytical devices (PAD) that can test for the presence of β-lactamase-mediated resistance as a form of AMR. To demonstrate viability, the PAD was used to detect β-lactam resistance in wastewater and sewage, and identified resistance in individual bacteria species isolated from environmental water sources.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the end-points of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. It will be further understood that the endpoints of each of the ranges are significant both in relation to the other endpoint, and independently of the other endpoint.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to bring about a recited effect, such as an amount necessary to form products in a reaction mixture. Determination of an effective amount is typically within the capacity of persons skilled in the art, especially in light of the detailed disclosure provided herein. The term “effective amount” is intended to include an amount of a compound or reagent described herein, or an amount of a combination of compounds or reagents described herein, e.g., that is effective to form products in a reaction mixture. Thus, an “effective amount” generally means an amount that provides the desired effect.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

The term “chromogenic indicator”, as used herein, refers to a substance that is chromogenic and may or may not be in solution with other reagents, solvents (e.g, water or an organic solvent), or buffers. The chromogenic indicator has a natural color (for example, yellow) that changes or shifts to another color (for example, red or shades between yellow and red) when a bond (other than a C—H bond) is broken (e.g., because of a chemical or enzymatic reaction), thereby changing the conjugation of electrons in the parent molecule.

The term “colony-forming unit” or “CFU”, refers to a unit used to estimate the number of viable bacteria, wherein “viable” refers to the ability of bacteria to multiply.

The term “portable”, as used herein, refers to a device that can be handheld, carried by a person without strain, or both.

The term “diffuser” or “light diffuser”, as used herein, refers to any material, that diffuses or scatters light evenly over a surface. For example, a diffuser can comprise, but is not limited to a translucent material, a glass, a polymer, white paper, or water.

### Embodiments of the Invention

This disclosure provides various embodiments of a system for beta-lactamase enzyme detection comprising:
a) a planar cellulose-based mesh comprising a first surface having a hydrophobic perimeter, a hydrophobic surface opposite the first surface, and a chromogenic indicator dispersed in the mesh within the hydrophobic perimeter; and
b) a portable digital imaging device that records color images;

wherein the imaging device records a color image of the chromogenic indicator, wherein a beta-lactamase enzyme is detected by a change in the color of the chromogenic indicator when in contact with a beta-lactamase enzyme.

In other embodiments, the planar cellulose-based mesh comprises filter paper or absorbent paper, and the hydrophobic perimeter comprises a wax. In other embodiments, the hydrophobic perimeter is paraffin. In yet other various embodiments, the chromogenic indicator comprises nitrocefin.

In additional embodiments, the portable digital imaging device comprises a smartphone and a container that is impenetrable to visible light. In some embodiments, the portable digital imaging device comprises a camera. In some other embodiments, the system comprises a light diffuser.

In other additional embodiments, the planar cellulose-based mesh comprises a blank hydrophobic perimeter, wherein the blank hydrophobic perimeter refers a hydrophobic permitted than does not have any substance within the perimeter other than the planar cellulose-based mesh (i.e., it is blank until, for example, a control or sample is added).

In yet other embodiments, the hydrophobic perimeter is an array of hydrophobic perimeters. In additional embodiments the said array is a matrix of m rows by n columns, wherein m is 1 or more and n is 1 or more. In other embodiments, m is 1 to 10,000 and n is 1 to 10,000, m is 1 to 1000 and n is 1 to 1000, or m is 1 to 100 and n is 1 to 100.

This disclosure also provides various embodiments of a method of detecting antimicrobial resistant (AMR) bacteria with the system disclosed above, comprising:
a) contacting a water sample with the chromogenic indicator dispersed in the mesh within the hydrophobic perimeter to form a mixture in the mesh;
b) incubating the mixture;
c) recording the color of the chromogenic indicator; and
d) analyzing the chromogenic indicator for a color change;

wherein a beta-lactamase enzyme from AMR bacteria that expresses the beta-lactamase enzyme is detected in the water sample by the change in the color of the chromogenic indicator relative to a control sample within a blank hydrophobic perimeter when the chromogenic indicator is contacted by the beta-lactamase enzyme.

In various additional embodiments, the chromogenic indicator dispersed in the mesh within the hydrophobic perimeter has been dried prior to contacting a water sample. In other embodiments, the control sample is purified water. In other additional embodiments, the color of the chromogenic indicator is recorded with a light diffuser over the planar cellulose-based mesh, the chromogenic indicator, or the control sample.

In various other embodiments, analyzing the chromogenic indicator for a color change comprises normalizing the color image of the chromogenic indicator by the control sample. In yet other embodiments, normalization comprises, analyzing the color image or light intensity of the chromogenic indicator (which may have contacted AMR bacteria), then subtracting the color image or light intensity of the control or purified water sample (or subtracting the average result from several controls). Thus, subtracting the light intensity of the control from the sample's light intensity normalizes the data and reduces the standard deviation when analyzing the chromogenic indicator for a color change.

In some embodiments, the area within the hydrophobic perimeter is less than about 100 mm². In other embodiments, the amount of the chromogenic indicator dispersed in the mesh within the hydrophobic perimeter is about 1 nanomole to about 10 nanomoles. In yet other embodiments, the amount of the chromogenic indicator is 1 picomole to 1 nanomole, 1 nanomole to 100 nanomoles, 50 nanomoles to 500 nanomoles, 500 nanomoles to 1 micromole, or 1 micromole to 100 micromoles.

In additional embodiments, the limit of detection (LOD) of AMR bacteria is about 1×10⁵ CFU/mL to about 1×10⁷ CFU/mL. In other embodiments the LOD is about 1×10⁴ CFU/mL, about 1×10⁵ CFU/mL, about 1×10⁶ CFU/mL, about 1×10⁷ CFU/mL, about 1×10⁸ CFU/mL, or about 1×10⁹ CFU/mL.

In other embodiments, bacteria in the water sample is lysed prior to step a). In yet other embodiments, the accuracy of detecting the presence of AMR bacteria in the water sample is greater than 95%. In some other embodiments, the accuracy is about 90% to about 99.99%, about 96%, about 97%, about 98%, about 99%, or about 99.5%.

Additionally, this disclosure provides various embodiments of a method for detecting beta-lactamase enzyme comprising:
a) drying one or more aliquots of a nitrocefin indicator on a sheet of absorbent paper comprising a first surface, one or more hydrophobic perimeters at the first surface, and a hydrophobic surface opposite the first surface, wherein a dried aliquot of the nitrocefin indicator is dispersed in the paper within the hydrophobic perimeter;
b) contacting a sample with the dried aliquot of the nitrocefin indicator to form a mixture in the paper; and
c) incubating the mixture;

wherein a beta-lactamase enzyme in a sample comprising the beta-lactamase enzyme is detected by the change in the color of the nitrocefin indicator relative to a control sample when the nitrocefin indicator contacts the beta-lactamase enzyme.

In some additional embodiments, the concentration of each aliquot of the nitrocefin indicator is about 0.1 mM to about 2 mM. In some other embodiments, the concentration is about 0.01 mM to about 0.1 mM, about 0.1 mM to about 0.5 mM, about 0.5 mM to about 1 mM, about 1 mM to about 1.5 mM, about 1.5 mM to about 2.0 mM, or about 1 mM to about 5 mM,

In yet other embodiments, the volume of each aliquot of the nitrocefin indicator that is dispersed in the paper within the hydrophobic perimeter is about 1 μL to about 10 μL. In some other embodiments, the volume is about 0.1 μL to about 1000 μL, about 0.1 μL to about 1 μL, about 0.5 μL to about 1 μL, about 1 μL to about 2 μL, about 2 μL to about 5 μL, about 5 μL to about 100 μL, about 0.2 μL, about 0.3 μL, about 0.4 μL, about 0.5 μL, about 0.6 μL, about 0.7 μL, about 0.8 μL, or about 0.9 μL.

In some additional embodiments, the nitrocefin indicator comprises a buffer. In other embodiments the buffer has a pH of about pH 5, about pH 5.5, about pH 6, about pH 6.5, about pH 7, about pH 7.5, about pH 8, about pH 8.5, about pH 9, about pH 9.5, about pH 10, or about pH 11.

In other embodiments, detection of the beta-lactamase enzyme has a limit of detection (LOD) of about 0.1 mU/mL to about 25 mU/mL. In other embodiments, the LOD is about 0.01 mU/mL to about 100 mU/mL, about 1 mU/mL to about 5 mU/mL, about 5 mU/mL to about 20 mU/mL, about 10 mU/mL to about 15 mU/mL, about 15 mU/mL to about 20 mU/mL, about 20 mU/mL to about 25 mU/mL, about 25 mU/mL to about 50 mU/mL, about 50 mU/mL to about 75 mU/mL, or about 75 mU/mL to about 100 mU/mL.

In various additional embodiments, the sample comprises blood, blood plasma, or antimicrobial-resistant bacteria, or a combination thereof. In other embodiments the sample comprises water.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

### Results and Discussion

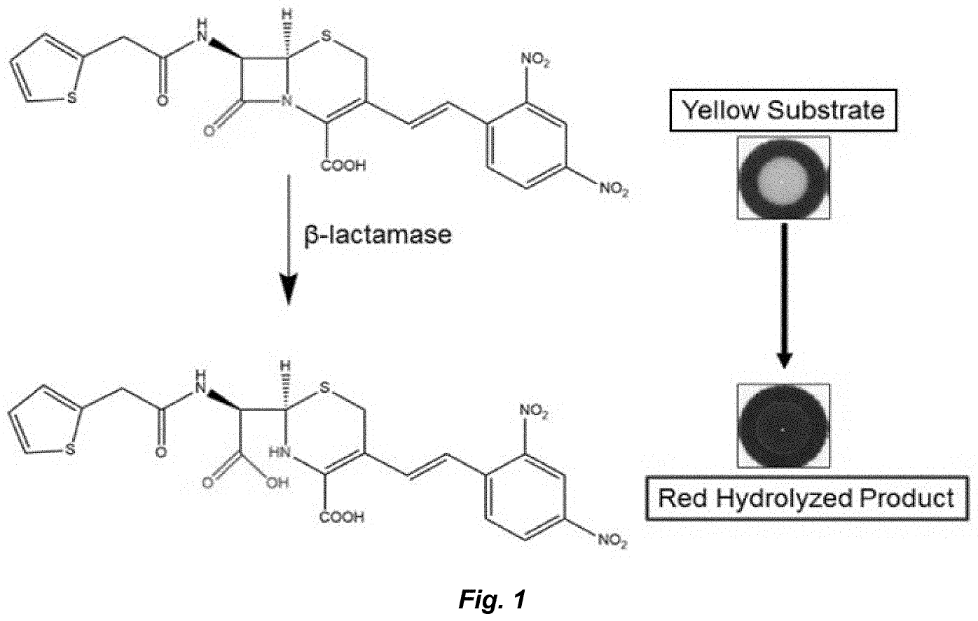

β-lactam antibiotics are the most widely used class of antibiotics. Bacterial resistance to these antibiotics are the most commonly acquired resistance classified as a serious threat by the Center for Disease Control (CDC). Resistance can be a result of bacterial expression of β-lactamase enzymes, which inactivate β-lactams by hydrolyzing the β-lactam ring in the antibiotic. Several ways exist to detect β-lactamase activity including reactions with nitrocefin, a chromogenic cephalosporin. The reaction results in the hydrolysis of the carbon-nitrogen bond in the β-lactam ring, causing a distinct color change from yellow to red (FIG. 1). Using this straightforward detection method, we have developed a PAD that can detect β-lactamase-expressing bacteria in real-world samples. The platform is inexpensive, costing ˜$0.20US per test, compared to $10-22US for antibiotic susceptibility testing, and provides sensitivity that matches that of a microtiter plate.

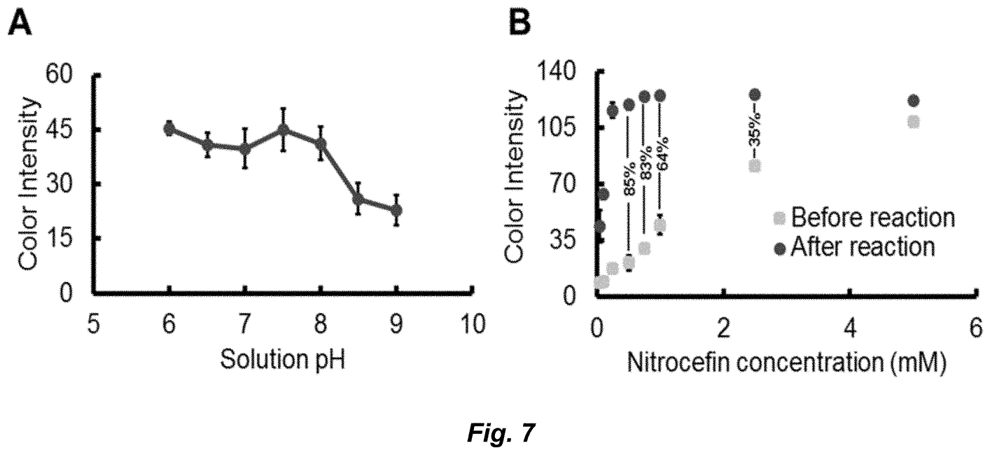

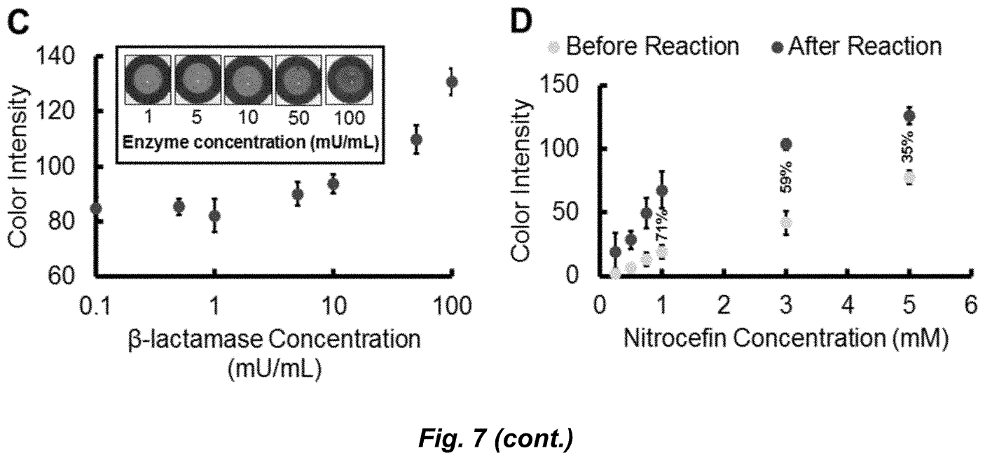

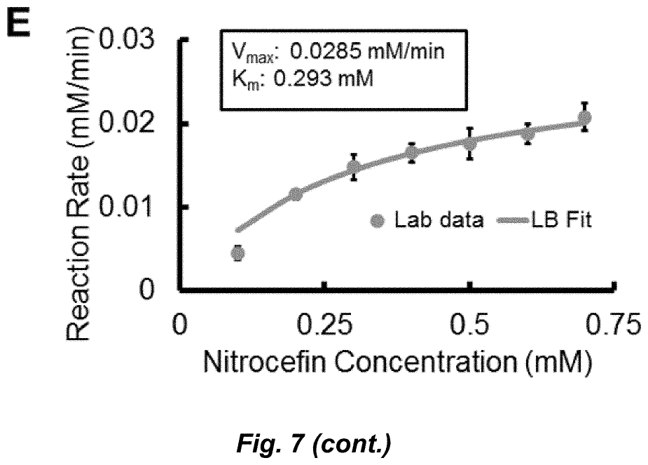

Reaction optimization between β-lactamase and nitrocefin was performed using arrays of 8-mm-diameter paper wells fabricated with Whatman #4 filter paper. The devices were photographed with a smartphone camera and analyzed with ImageJ software. Phosphate buffered saline (PBS) was used in solution and the optimal reaction pH was determined to be pH 7.5. The optimal nitrocefin concentration was 0.5 mM to maximize product signal, and the limit-of-detection (LOD) of lyophilized β-lactamase was 10 mU/mL. Additional details on reaction optimization is provided in the Example section below. The Michaelis-Menten kinetics of β-lactamase and nitrocefin were calculated for reactions on paper at ˜22° C. Using a Lineweaver-Burk plot, the calculated Vₘₐₓ was 0.0285±0.0012 mM/min and Kₘ was 0.293±0.013 mM (FIG. 7E). Literature searches have not generated published Michaelis-Menten values for β-lactamase reacting with nitrocefin, but were similar to other reported values for β-lactamase. This similarity in Michaelis-Menten values exhibits promise for the paper-based assays.

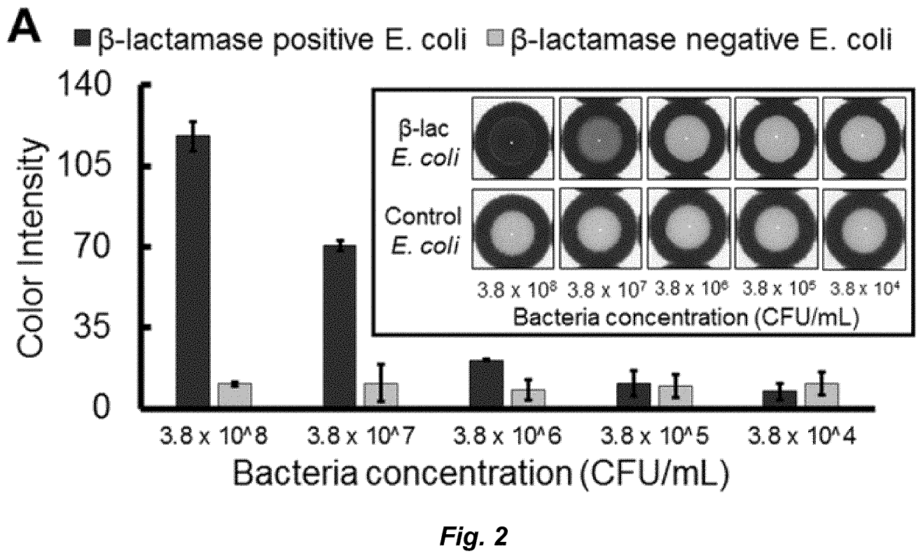

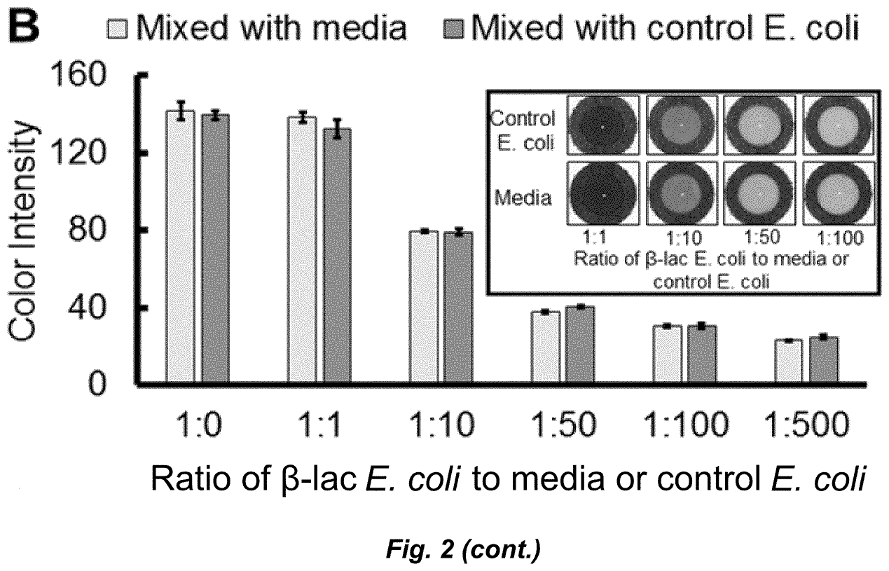

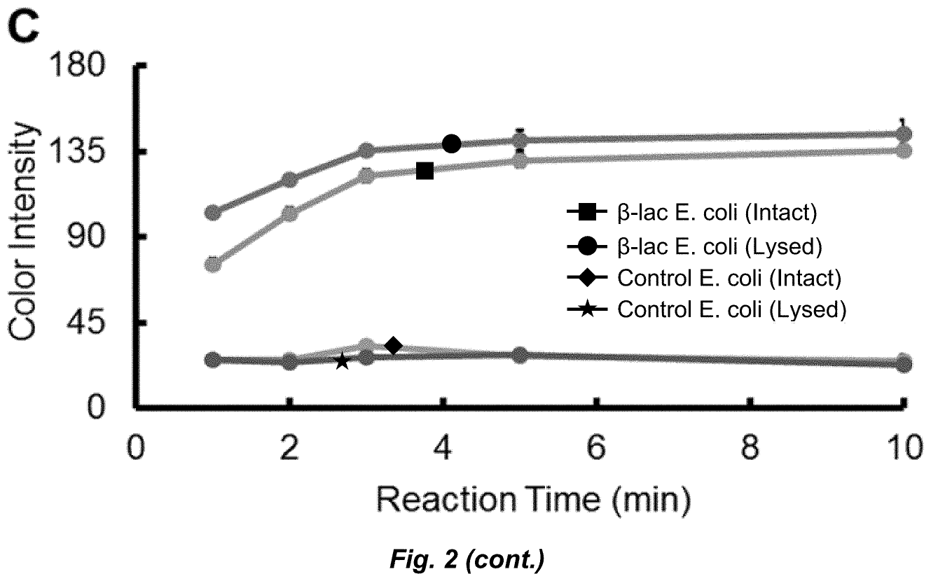

To demonstrate detection of β-lactamase in live bacteria, the optimized reaction conditions were used to analyze E. coli without culturing. Serial dilutions of β-lactamase-expressing E. coli and control E. coli were reacted with 0.5 mM nitrocefin at room temperature directly on the paper devices. No color change was observed unless the bacteria expressed β-lactamase (FIG. 2A). The color change in the assay occurred with more than 3.8×10⁶ CFU/mL bacteria, but not with lower concentrations. To determine whether non-β-lactamase producing bacteria would interfere with the detection of β-lactamase-producing E. coli, different ratios of β-lactamase-expressing bacteria to control bacteria were analyzed. The color intensities were the same with or without non-β-lactamase producing bacteria present in the sample (FIG. 2B). E. coli that do not express β-lactamase do not interfere with the reaction as similar color intensities were observed in pure or mixed cultures. β-lactamase is produced within bacteria, so to attempt increasing sensitivity, we repeated the assay with lysing. For DH5α E. coli cells expressing β-lactamase, lysing the cells using probe sonication helped obtain a faster and more intense signal, but only marginally compared to no lysing (FIG. 2C). After 10 min of reaction, the color intensity of lysed cells was approximately 5% higher than intact cells. These results indicate the cells either secrete β-lactamase or nitrocefin is cell permeable. Several studies support bacteria translocating β-lactamase from the cytoplasm across the bacteria's inner membrane into the periplasm, but not outside the cell entirely, supporting the latter hypothesis.

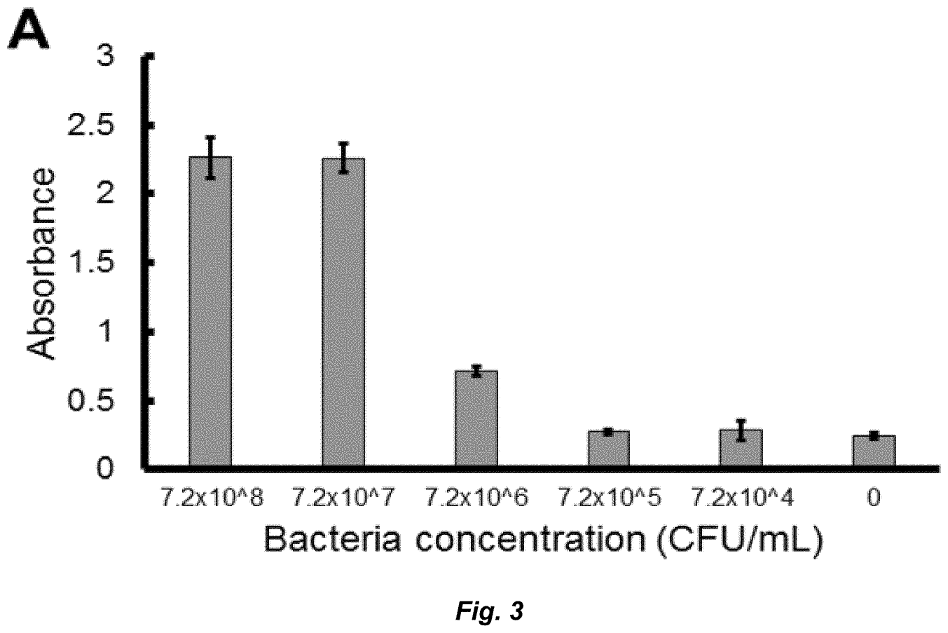

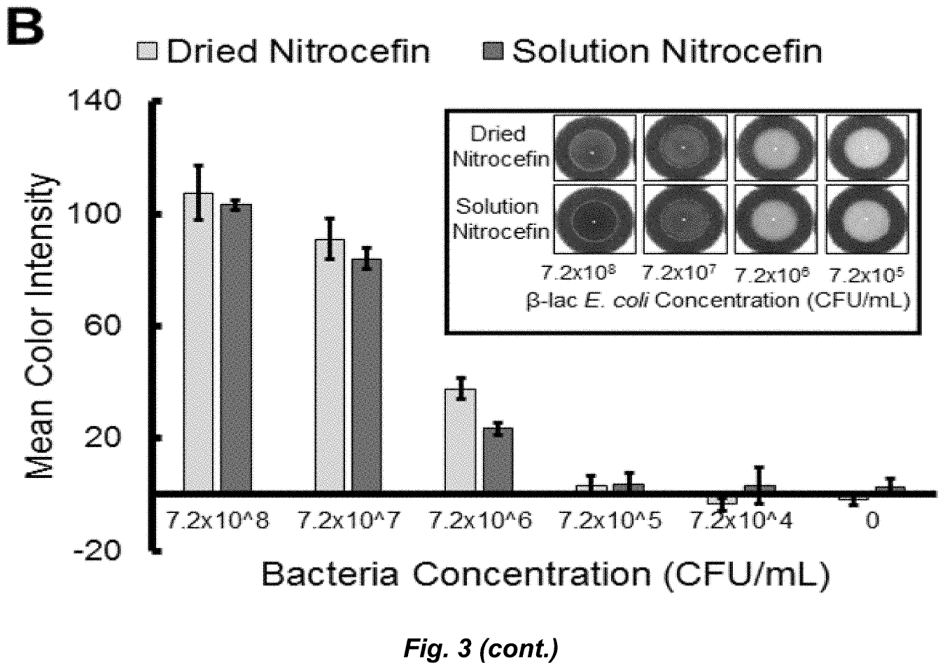

Because nitrocefin is a colorimetric substrate, it has been speculated whether using UV-visible spectrophotometry would result in more sensitive bacteria detection. Serial dilutions of laboratory E. coli expressing β-lactamase were reacted in a microtiter plate with nitrocefin and the absorbance was measured using a plate reader. Using a microtiter plate and plate reader compared to a PAD and smartphone did not yield a lower LOD (FIG. 3A). This demonstrates that using a PAD and smartphone is a cost-effective way to detect bacteria using nitrocefin, without the need for expensive instrumentation. Because the goal of point-of-need devices is to have a final product that can be taken into the field with minimal supplies for testing, it was also investigated whether nitrocefin could be dried in the paper beforehand. It was determined that the ideal concentration to dry into paper was 5 μL of 1 mM nitrocefin. Adding 40 μL of bacteria sample to the PAD test with dried nitrocefin was compared to PAD tests that held 20 μL of 0.5 mM nitrocefin solution and 20 μL of bacteria sample. Drying nitrocefin onto the paper before adding the sample showed slightly more sensitive results compared to nitrocefin solution (FIG. 3B). This is likely because nitrocefin did not need to be added to the total solution volume, therefore more sample could be added.

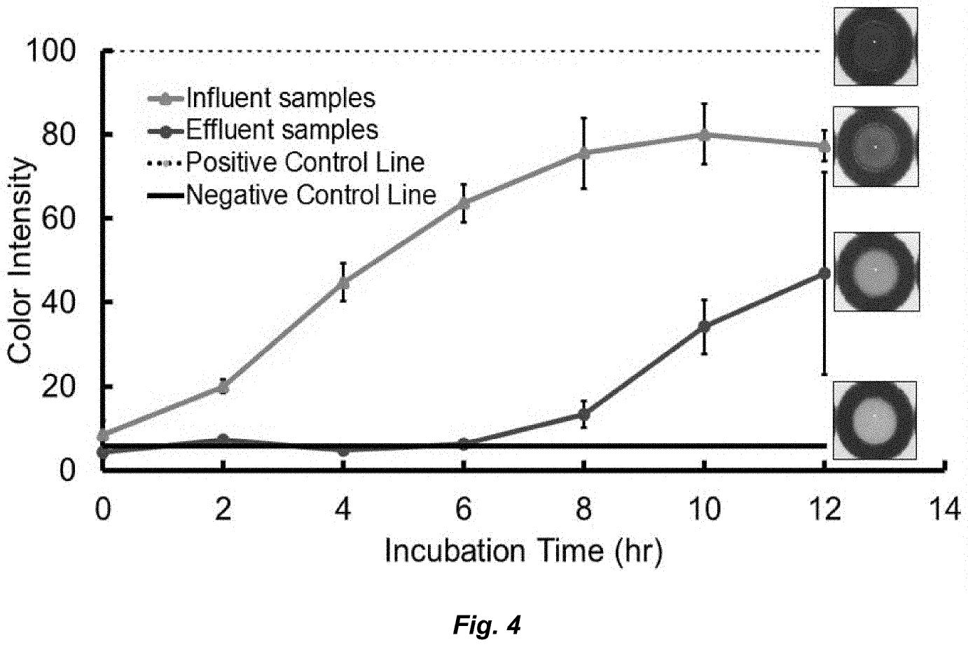

To confirm the new method would work with real-world samples, influent and effluent water was obtained from the Drake Water Reclamation Facility located in Fort Collins, Colo., United States. In the influent, β-lactamase was detected after only 2 hr of sample incubation in media. The signal continually increased until reaching a maximum at ˜10 hr of incubation (FIG. 4). Similar results could be obtained with a microtiter plate but at much higher costs. The effluent, which should contain less bacteria, did not show a signal until 8 hr of incubation. These results were confirmed using dilution and plating methods, which gave a concentration of 4.50×10⁶ CFU/mL of total bacteria in the influent, and 5.08×10³ CFU/mL of total bacteria in the effluent. AMR bacteria were confirmed using commercially available extended-spectrum-β-lactamase (ESBL) plates from CHROMagar™. On these plates, there were 4.96×10⁴ CFU/mL of total ESBL-containing bacteria in the influent and 1.30×10¹ CFU/mL in the effluent. This correlates to 1.1% and 0.257% ESBL bacteria in the influent and effluent respectively. The high signal that was obtained in the influent sample, considering a 1:99 ratio of β-lactam-resistant bacteria to non-resistant bacteria, could be due to several factors. First, bacteria resistant to β-lactam antibiotics could be growing at a faster rate compared to non-resistant bacteria, therefore occupying more of the sample once it was concentrated enough to detect resistance. This variance in growth rate was also observed in the effluent between samples as demonstrated by the large error bars at 12 hr. The sewage sample bacteria also had to react for over an hour with nitrocefin to obtain a detectable signal, compared to 2-5 min of reaction for samples that were entirely resistant bacteria. However, this slower reaction rate could also be due to chemicals in the sewage water interfering with the enzymatic reaction.

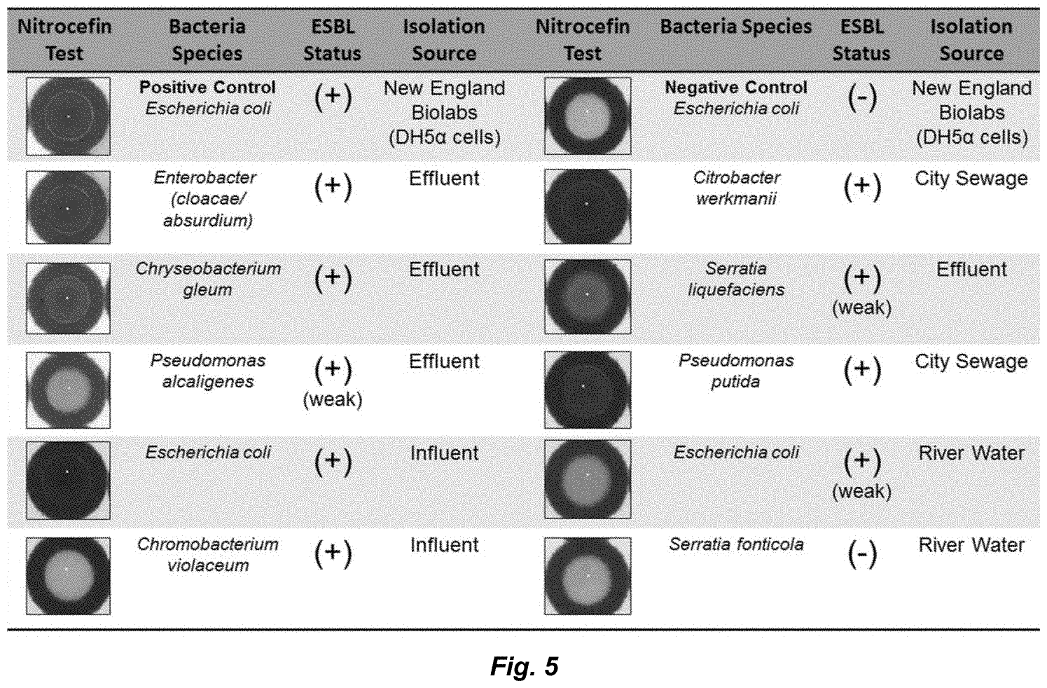

In order to determine how many different bacterial species were detected in the sewage samples, several bacteria species were isolated and cultured from the original sewage and other environmental samples. The bacteria cultures were given to the tester blind to ensure no biases when using the paper-based tests. Of 10 different bacteria isolates tested from a variety of species and environmental sources, there were no false positives and one false negative (FIG. 5). Bacteria solutions were kept intact and not lysed for consistency. When using the paper-based test on intact bacteria, results indicate that the assay could also quantify resistivity for different bacteria species. The “slightly positive” paper tests corresponded to “weak positives” that were confirmed via CHROMagar™ ESBL plates. “Slightly positive” was defined as having a color intensity change of 20%-80% compared the positive control laboratory E. coli, and “weak positive” was defined as reduced bacteria growth on ESBL plates compared to a non-antibiotic plate.

One bacterial isolate, Chromobacterium violaceum, tested negative using the paper-based test but tested positive using a CHROMagar™ ESBL plate. This same species did not grow on an ampicillin-containing agar plate, indicating that it is likely susceptible to penicillins. To confirm which test was correct, the minimum inhibitory concentration (MIC) of different β-lactam antibiotics was tested. The isolate was resistant to cephalosporins like cefazolin and cephalothin, but was susceptible to penicillins, such as amoxicillin and ticarcillin. The bacteria were also susceptible to imipenem, a carbapenem β-lactam antibiotic that is used as a last resort in clinical cases. Overall, this resistance profile is similar to a previously reported profile exhibiting resistance to cephalosporins, but sensitivity to penicillins. Nitrocefin did not react with C. violaceum's β-lactamase possibly because of an inhibitor in the sample. Nitrocefin is defined as a chromogenic cephalosporin, so nitrocefin is generally expected to be reactive with a β-lactamase that protects the cell against cephalosporin antibiotics.

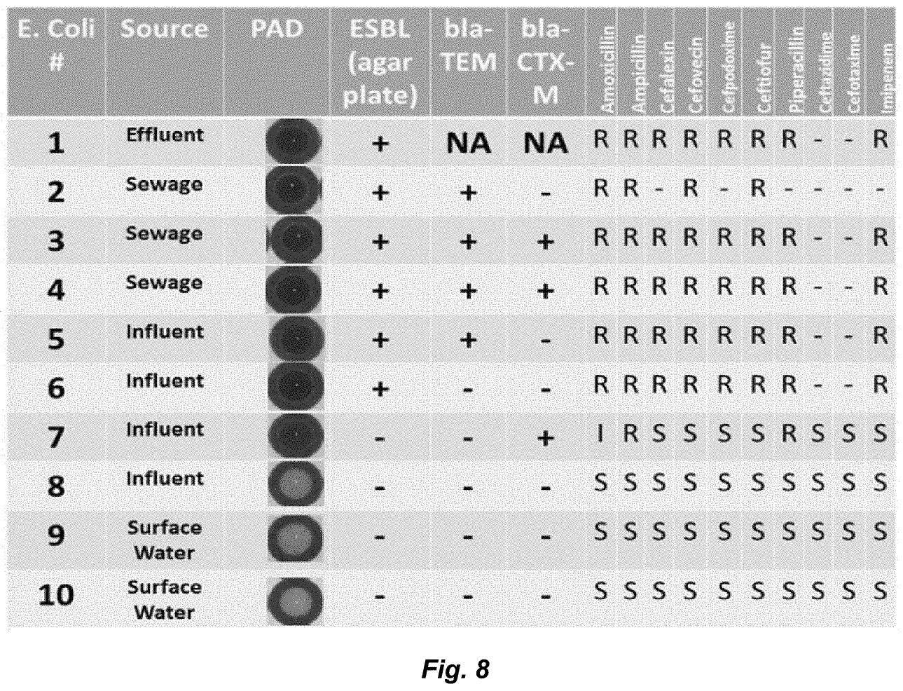

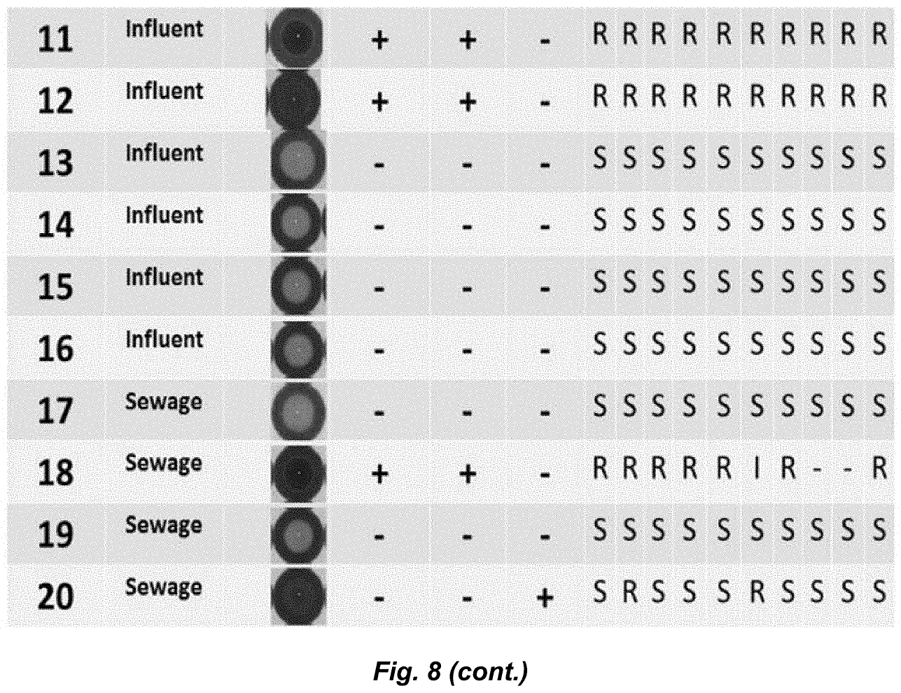

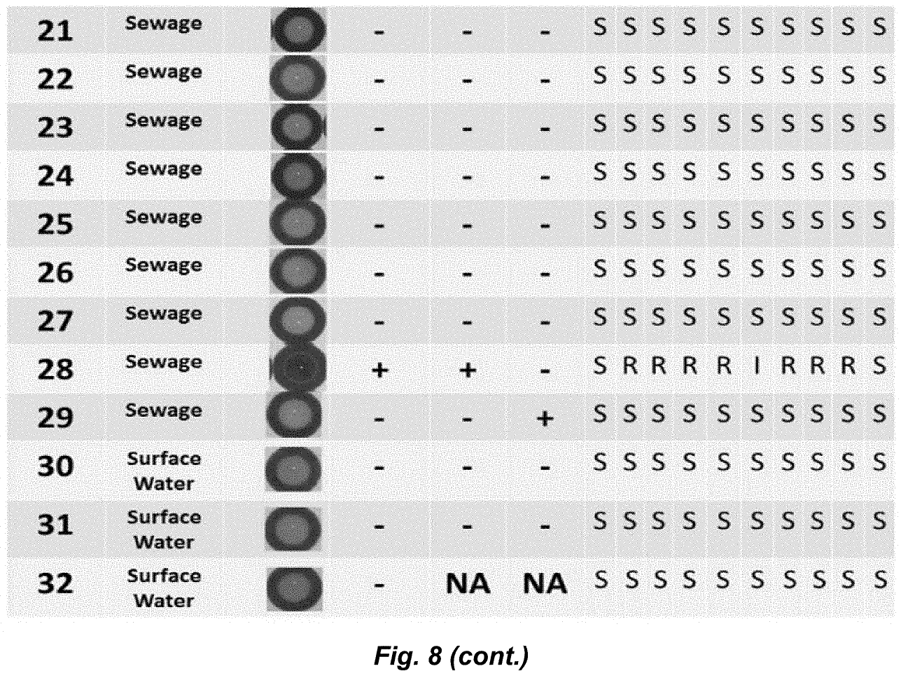

While ESBL-selecting plates are a common method to determine β-lactamase expression, it is more common in medicine to subject bacteria to antibiotic susceptibility testing. To compare the PAD to this method, 32 different environmental E. coli isolates were subjected to antibiotic susceptibility testing of different β-lactam antibiotics as well as plating the isolates on ESBL-selecting plates. The PAD test was compared to these methods for accuracy, and no false negatives were observed (FIG. 8). When comparing the PAD test to ESBL-selecting plates, two false positives occurred (isolate #7 and #20). However, when comparing to antibiotic susceptibility testing, these isolates were resistant to at least two penicillin antibiotics. When comparing the PAD test to antibiotic susceptibility testing, the tests were negative when the bacteria were susceptible to all tested antibiotics, and were positive when resistant to any of the tested antibiotics. As further confirmation, the E. coli isolates were also tested for the presence of ESBL genes blaTEM and blaCTX-M using polymerase chain reaction (PCR). Isolates #7 and #20 had the blaTEM gene present in their genome, also corresponding to the PAD results (FIG. 8). With 42 tested isolates and one true false negative, this test has so far shown 97.6% accuracy.

A straightforward and accurate paper-based colorimetric assay to detect bacteria resistant to β-lactam antibiotics has been developed that costs ˜$0.20 per test but gives similar sensitivity to more expensive microtiter plate methods. We have also optimized the enzymatic reaction between nitrocefin and β-lactamase on paper, and demonstrated that non-AMR bacteria do not interfere with the assay performance and cell lysis is not required. Detecting β-lactamase-expressing bacteria in community sewage water and identifying resistance in various species of bacterial isolates has demonstrated the practicality of this method. All tests were confirmed and compared to traditional culturing methods, antibiotic susceptibility testing, and PCR gene analysis. Although a laboratory was necessary to concentrate the sewage samples, this method still reduced the laboratory process by 14-20 hr. This test is also possible to ultimately integrate into a field-ready module by creating a more sensitive test or concentrating samples in the field. Bacterial samples were shown to react with nitrocefin whether in solution or dried into the paper, also demonstrating its potential for a field-ready module. It was confirmed that using a paper-based test and a camera phone for quantification yielded the same LOD as using an expensive and non-transportable plate reader and microtiter plate. While traditional methods are also quantitative of resistance, our paper-based method would be a rapid, cost-effective surveillance tool with a yes/no informed decision outcome prior to establishing a need for additional testing.

The following Example is intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLE

### Example 1. Materials and Methods

### Device Fabrication and Data Analysis

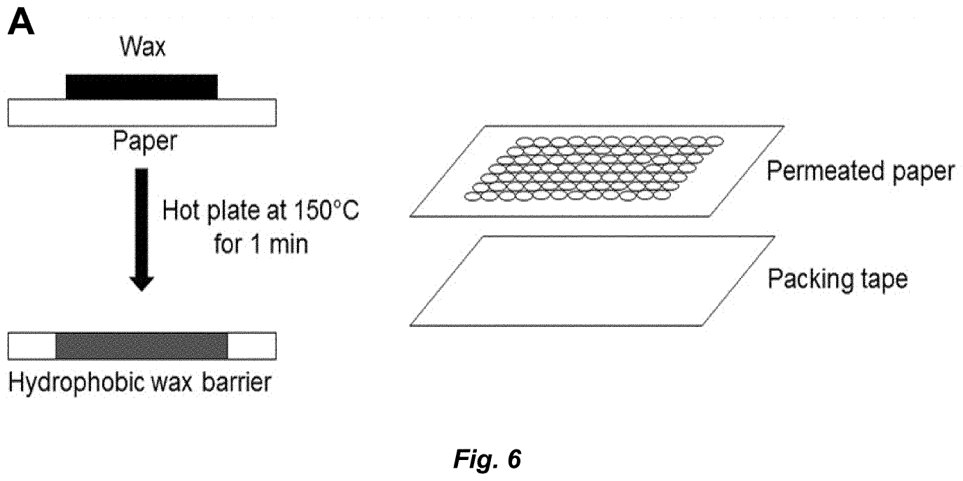

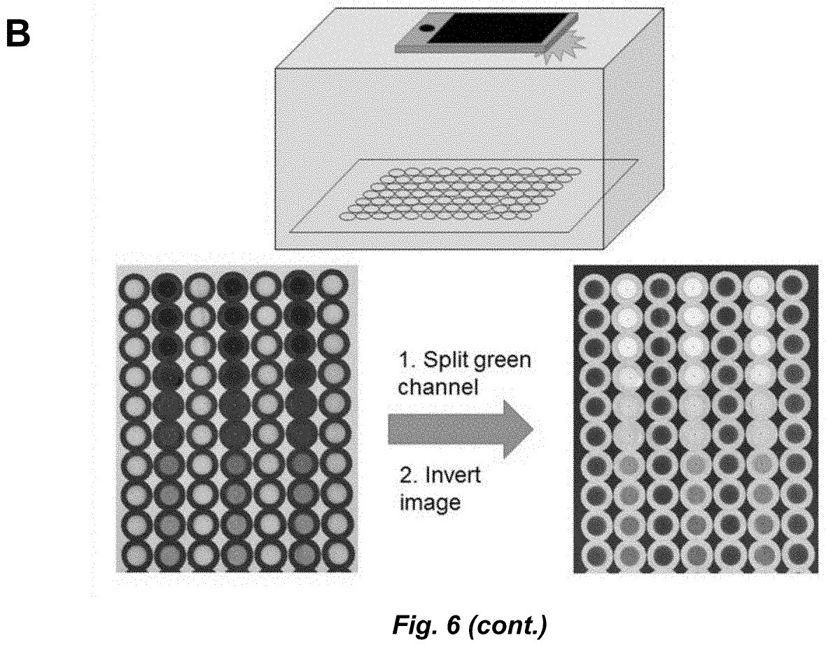

The devices were fabricated with Whatman chromatography paper grade 4 [GE Healthcare Life Sciences], using a simple design of black circles on a 7 by 12 grid designed with CorelDraw X4. Whatman 4 was selected based on separate enzyme kinetics experiments. It was determined in this separate project that Whatman 4, due to its larger pores, therefore less surface area, has less nonspecific adsorption, results in a higher colorimetric signal. Each device circle was designed with a 4 pt line and measured 8×8 mm. To define the device's hydrophobic barriers, a ColorQube 8870 [Xerox] wax printer was used to dispense wax on the surface. An IsoTemp [Fisher Scientific] hot plate was set to 150° C. with two metal plates and wax-printed paper was placed between the hot plate and a metal plate for 1 min to allow wax to melt through the pores. Scotch Shipping Heavy Duty packing tape was then taped on the back of the paper to prevent sample leakage (FIG. 6A).

To make devices with nitrocefin dried into the paper before reaction, 5 μL of nitrocefin solution was dried into the chromatography paper before taping the back with packing tape. Devices were dried at 4° C. because it was determined that drying at lower temperatures away from light resulted in more efficient reactions with samples.

For quantifying colorimetric products, a “light box” and the camera of an iPhone 5C or 5S were used to capture images and send to computer for image analysis (FIG. 6B). The resolution on an iPhone 5C and 5S are reported to be 8 megapixels with a resolution of 3264×2448. Using this method, we could obtain kinetic results as opposed to simply endpoint results that would be obtained using an office scanner. Pictures were taken within the box (measured 16×16×16 cm) designed to encompass the entire paper analytic device and to limit any outside light. In order to capture the image, a slit measured 2×5 cm was cut out of the top to allow a view inside the box for the camera phone and flash. The box interior was lined with standard white copy paper to best disperse light from the camera's flash. For each experiment, three samples of each reaction were placed in every other column of circles. Water was placed in the columns on each side of the samples to act as a “light control.” Due to the imperfect flash intensity across the paper, the light controls were used to normalize the intensity of each sample spot to give more precise results.

Images were sent to a computer and analyzed using NIH ImageJ software. The image was split into its color channels and the green color channel was selected and inverted. The green channel was selected because it is the complimentary color of red, the reaction's endpoint color. The color intensity of each sample spot was quantified, then normalized by subtracting the mean intensity of the water spots on each side of the sample spot. Normalized values were input into Microsoft Excel where the mean and standard deviation of samples were obtained. Standard deviation was represented in statistical graphs as error bars.

Characterization of β-lactamase and Nitrocefin Reaction

Nitrocefin [VWR International], a chromogenic cephalosporin, was used for detection of β-lactamase because of the distinct color change from yellow to red in the presence of the enzyme, making it a user-friendly platform. 5 mg of nitrocefin was initially dissolved in 1 mL dimethylsulfoxide (DMSO), because the substrate is insoluble in water. Aliquots of 9.68 mM nitrocefin was frozen at −20° C. in amber microcentrifuge tubes [VWR International]. These tubes were used to minimize degradation from UV exposure. Aliquots were taken out and allowed to thaw and warm to room temperature. Nitrocefin was further diluted with pH 7.4 phosphate buffered saline (PBS) [1.37 M NaCl, 0.027 M of KCl, 0.1 M Na₂HPO₄, and 0.018 M KH₂PO₄] to a concentration of 0.5 mM for each experiment (except for nitrocefin concentration optimization where 0.5 mM was selected). During pH optimization experiments, nitrocefin was diluted in pH buffers ranging from pH 6 to pH 9. Recombinant β-lactamase was purchased at a concentration of 1,500 U/mg [Abcam] and was initially dissolved in dH₂O and aliquoted and frozen. It was diluted with PBS before optimization experiments. For each reaction, 20 μL of nitrocefin would react with 20 μL of β-lactamase. Images were obtained at 2 hr, to ensure reaction completion. For determining the limit of detection of β-lactamase, the image was taken at 4 hr.

β-lactamase Kinetics

In order to quantify the concentration of nitrocefin that was hydrolyzed every minute, a calibration curve was generated by quantifying the red product after the reaction had completed, and plotting green light intensity vs. product concentration. The linear regression equation of this line was used to calculate the product concentration in the solution at each time point. The slope of the line of red intensity between 3 and 5 min was used to calculate the reaction rate. Eight different concentrations of nitrocefin was used to obtain a kinetic curve (0.1, 0.2, 0.3, 0.4, 0.5, 0.6, and 0.7 mM). To obtain Vₘₐₓ and Kₘ, a Lineweaver-Burk plot was generated by plotting 1/[S] vs. 1/v and the inverse x- and y-intercepts were obtained. The calculated Vₘₐₓ and Kₘ values were carried out in the Michaelis-Menten equation to obtain a theoretical kinetic curve to compare to data points.

### Live Bacteria Detection

DH5α E. coli cells [New England Biolabs] were used for both control and experimental bacteria in initial laboratory bacterial analysis. The control E. coli did not express β-lactamase, while the experimental bacteria expressed a previously published plasmid, pBG143, which encodes β-lactamase (Journal of Molecular Biology 2009, 385, 1643). The pBG143 plasmid was transformed into DH5α cells and the bacteria were incubated in Luria broth (LB) media containing 50 μg/mL ampicillin to select bacteria exclusively expressing β-lactamase. For subsequent experiments, bacteria were cultured in LB media overnight (˜12-16 hr) before each experiment. To determine our limit of detection, bacteria concentration was calculated using serial dilution and plating 50 μL of 10⁻⁶ and 10⁻⁷ dilutions on LB Agar plates containing no antibiotics and allowed to grow for 24 hr. The colonies were counted on each plate, calculated back to the original concentration, and the average was taken to obtain an estimate of the original bacteria concentration. To determine whether cell lysis was necessary for β-lactamase detection, the cells were sonicated for 20 sec using a XL-2000 Series probe sonicator set at 5 W, 22 kHz. The sonication settings and time was selected due to previously published data on sonicating E. coli cells (Anal Chem 2012, 84, 2900). Similar to optimizing the reaction with pure enzyme, 20 μL of bacterial culture was reacted with 20 μL of 0.5 mM nitrocefin and images were obtained after 2 hr of reaction. For all experiments involving bacteria, devices were placed in a petri dish to help prevent outside contamination and evaporation during the reaction time.

To detect bacteria using UV-vis spectrophotometry, a PerkinElmer Victor X5 multilabel plate reader was used to measure the change in absorbance in a microtiter plate. 100 μL of nitrocefin was mixed with 100 μL of bacteria sample and allowed to react for 2 hr when the absorbance was taken at 490 nm. The microtiter plate was covered with a plate lid to prevent evaporation during reaction.

Detecting β-lactamase in Sewage Samples

Waste water samples were collected from the Drake Water Reclamation Facility located in Fort Collins, Colo., United States [Collected on Sep. 7, 2016 at approximately 10 a.m.]. Influent samples were collected using a Hawk Composite Sampler, and effluent samples were collected as a grab sample post SO₂ treatment. After collection, influent and effluent samples were put directly on ice away from light for transportation back to the laboratory. 1 mL of sample was mixed with 3 mL of LB media and incubated in a 37° C. shaker. Three samples were taken of each the influent and effluent. Sample solution was taken out of the incubator every 2 hr to test for the presence of β-lactamase activity by reacting 20 μL of sample with 20 μL of 0.5 mM nitrocefin for 2 hr, when a picture would be obtained and analyzed. 0 hr samples were not mixed with media, but were reacted directly with nitrocefin.

Results were confirmed by membrane [0.45 μm mixed cellulose esters millipore membrane, MilliporeSigma™] filtration techniques on Orientation plates and extended-spectrum-β-lactamase (ESBL) selecting plates [CHROMagar™]. Influent sewage samples were diluted through 10-fold dilutions, and the 10⁻⁶, 10⁻⁵, and 10⁻⁴ dilutions were plated on ESBL and Orientation plates and allowed to grow at 37° C. for 24 hr. The colony forming units were counted and calculated to CFUs/100 mL. Relative percentage of resistant bacteria was calculated with Orientation (non-selective) as the denominator and ESBL (selective) as the numerator from the same source.

Obtaining and Testing Bacterial Isolates from Sewage and Environmental Samples

Bacterial isolates were obtained from grab samples in the field, except for influent. Field samples include influent, effluent, surface water from a river and sewage samples from city sewers. All samples were plated by pipetting 50-100 μL on various clinical agars [CHROMagar™ Orientation, CHROMagar™ ESBL, and CHROMagar™ KPC]. Bacterial isolates were purified by selecting a single colony with an inoculating loop and spreading the colony on the same kind of agar the colony was selected from, or was spread onto a MacConkey agar plate [Difco]. To remove potential inhibitors before any testing, they were further subcultured onto non selective agar [Tryptic Soy Agar, Thermo Scientific™ Remel™] and then grown in a nutrient broth [Tryptic Soy Broth, Thermo Scientific™ Remel™, Soybean Casein Digest] at 37° C. and 2% CO₂. After replenishing the nutrient broth, all bacteria isolates were grown using a shaker for 12-18 hr depending on bacteria growth rate. They were also re-plated on ESBL plates to confirm resistance mechanisms. The isolates were given to the tester blind for accurate, unbiased results. Bacteria were not lysed before reacting 20 μL of bacteria solution with 20 μL of 0.5 mM nitrocefin. Images were obtained after 2 hr of reaction.

Matrix-assisted laser desorption ionization time of flight mass spectrophotometry (MALDI-TOF) was used for speciation of isolates. Isolated bacterial cultures purified from selective media were sent to the Colorado State University Veterinary Teaching Hospital-Diagnostic Medical Center (Vet-DMC) to be analyzed. These samples were grown on blood agar plates and analyzed to identify species [VITEK-MS™ Biomerieux, USA]. Samples that could not be confidently identified at 99.9% or above by MALDI-TOF analysis were identified by 16-S-PCR of the variable 4 region.

To determine antibiotic susceptibility of Chromobacterium violaceum and the 32 E. coli isolates, each isolate was subjected to antibiotic susceptibility testing [VITEK 2™ Biomerieux, USA] using microdilution and photometric determination of growth at the Colorado State University Veterinary Diagnostic Laboratory located in Fort Collins, Colo., US. Minimum Inhibitory Concentration's (MICs) were reported in μg/mL, and results were interpreted per the Clinical Laboratory Standard Institute (CLSI). The antibiotics that were tested against C. violaceum included amikacin, amoxicillin-clavulanate, ampicillin, cefazolin, cefpodoxime, ceftazidime, cephalothin, imipenem, ticarcillin, and ticarcillin-clavulanate. Antibiotics tested against each E. coli isolate included amoxicillin, ampicillin, cefalexin, cefovecin, cefpodoxime, ceftiofur, piperacillin, ceftazidime, cefotaxime, and imipenem.

Polyermerase chain reaction (PCR) was also performed by the Colorado State University Veterinary Diagnostic Laboratory. These diagnostic tests were used to determine whether the bacterial isolates' genome contained ESBL genes blaTEM and/or blaCTX-M. PCR was performed using the diagnostic lab's standard procedure as follows. The following are the primer sequences used for the amplification of the isolated DNA: CTX-M (F: ATG TGC AGY ACC AGT AAR GTK ATG GC, R: TGG GTR AAR TAR GTS ACC AGA AYC AGC GG, 593 bp) and TEM (F: CGC CGC ATA CAC TAT TCT CAG AAT GA, R: ACG CTC ACC GGC TCC AGA TTT AT, 445 bp). 32 E. coli isolates from ChromAgar™ ESBL and ChromAgar™ Orientation were lysed in 100 μL of water per sample at 100° C. for 1 hr using BIO-RAD T100™ Thermocycler [Bio-Rad Laboratories, Inc, California]. Amplification was carried out by 2 μL DNA, 10 pmol of each primer, and 12.5 μl Emerald Amp® GT PCR Master Mix [Takara Bio Inc., Clontech, Japan] under conditions described by Amaya et al (Med. Microbiol., 2011, 60, 216). The PCR conditions were as followed: 15 minutes of denaturation at 95° C. (1 cycle), 30 seconds of denaturation at 94° C., 90 seconds of annealing at 62° C., and 1 minute of polymeration at 72° C. (34 cycles), with a final extension at 72° C. for 10 minutes. PCR products were analyzed on a 1.5% agarose gel [BioRad] and visualized using Ethidium Bromide (item). Single reaction PCR confirmed the presence or absence of each gene.

Optimization of the β-lactamase and Nitrocefin Reaction

Reaction optimization was performed using arrays of 8-min-diameter paper wells fabricated with Whatman #4 chromatography paper. In all studies, assays were kept at room temperature (˜22° C.) to best mimic field conditions. The devices were photographed, then analyzed with NIH Image software. To determine the optimal assay pH, β-lactamase and nitrocefin were reacted in phosphate buffered saline (PBS) solutions between pH 6 and pH 9 (FIG. 7A). PBS pH 6.0 and 7.5 displayed the highest reaction efficiency. Other tests were included at pH 7.4 (pH 7.0 for enzyme limit-of-detection) to best mimic blood pH for possible point-of-care diagnostic applications.

Optimal substrate concentration was determined using a constant concentration of β-lactamase (100 U/mL for prompt results) incubated with varying concentrations of nitrocefin. 1 mM nitrocefin provided the highest final color intensity, whereas 0.25 to 0.5 mM nitrocefin produced the largest color intensity change of 85-83% compared to a 64% intensity change observed with 1 mM nitrocefin (FIG. 7B). Using nitrocefin at a concentration above 1 mM results in a very dark starting sample solution, making changes in the reaction color difficult to measure. Hence, lower concentrations of nitrocefin are optimal for generating the widest dynamic range for detection of β-lactamase.

To find the limit of β-lactamase enzyme detection, the minimum concentration of β-lactamase present that could react with nitrocefin to give a measurable color change was established. 0.5 mM nitrocefin was reacted with decreasing concentrations of recombinant β-lactamase for 4 hr and imaged. The enzyme showed little difference in light intensity at lower concentrations (FIG. 7C). Any concentration of β-lactamase lower than 10 mU/mL, does not show enough color intensity to be detected accurately.

The optimal nitrocefin concentration to dry into the paper was determined by drying 5 μL of different concentrations of nitrocefin on chromatography paper and observing the change in color intensity before and after adding 1 U/mL of β-lactamase for 30 min. Similar to nitrocefin in solution, too high of concentrations of nitrocefin resulted in too dark of a starting spot, thus 1 mM was determined to be the optimal concentration to dry on paper with a color intensity change of 71% (FIG. 7D).

FIG. 7E displays the Michaelis-Menten curve for the reaction between β-lactamase and nitrocefin, which was described above.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

1. A system for beta-lactamase enzyme detection comprising:
a) a planar cellulose-based mesh comprising a first surface having a hydrophobic perimeter, a hydrophobic surface opposite the first surface, and a chromogenic indicator dispersed in the mesh within the hydrophobic perimeter; and
b) a portable digital imaging device that records color images;
wherein the imaging device records a color image of the chromogenic indicator, wherein a beta-lactamase enzyme is detected by a change in the color of the chromogenic indicator when in contact with a beta-lactamase enzyme.

2. The system of claim 1 wherein the planar cellulose-based mesh comprises filter paper or absorbent paper, and the hydrophobic perimeter comprises a wax.

3. The system of claim 2 wherein the chromogenic indicator comprises nitrocefin.

4. The system of claim 1 wherein the portable digital imaging device comprises a smartphone and a container that is impenetrable to visible light.

5. The system of claim 4 wherein the planar cellulose-based mesh comprises a blank hydrophobic perimeter.

6. The system of claim 1 wherein the hydrophobic perimeter is an array of hydrophobic perimeters.

7. A method of detecting antimicrobial resistant (AMR) bacteria with the system of claim 1 comprising:
a) contacting a water sample with the chromogenic indicator dispersed in the mesh within the hydrophobic perimeter to form a mixture in the mesh;
b) incubating the mixture;
c) recording the color of the chromogenic indicator; and
d) analyzing the chromogenic indicator for a color change;
wherein a beta-lactamase enzyme from AMR bacteria that expresses the beta-lactamase enzyme is detected in the water sample by the change in the color of the chromogenic indicator relative to a control sample within a blank hydrophobic perimeter when the chromogenic indicator is contacted by the beta-lactamase enzyme.

8. The method of claim 7 wherein the chromogenic indicator dispersed in the mesh within the hydrophobic perimeter has been dried prior to contacting a water sample.

9. The method of claim 7 wherein the control sample is purified water.

10. The method of claim 7 wherein analyzing the chromogenic indicator for a color change comprises normalizing the color image of the chromogenic indicator by the control sample.

11. The method of claim 7 wherein the area within the hydrophobic perimeter is less than about 100 mm².

12. The method of claim 11 wherein the amount of the chromogenic indicator dispersed in the mesh within the hydrophobic perimeter is about 1 nanomole to about 10 nanomoles.

13. The method of claim 7 wherein the limit of detection of AMR bacteria is about 1×10⁵ CFU/mL to about 1×10⁷ CFU/mL.

14. The method of claim 7 wherein bacteria in the water sample is lysed prior to step a).

15. The method of claim 7 wherein the accuracy of detecting the presence of AMR bacteria in the water sample is greater than 95%.

16. A method for detecting beta-lactamase enzyme comprising:
a) drying one or more aliquots of a nitrocefin indicator on a sheet of absorbent paper comprising a first surface, one or more hydrophobic perimeters at the first surface, and a hydrophobic surface opposite the first surface, wherein a dried aliquot of the nitrocefin indicator is dispersed in the paper within the hydrophobic perimeter;
b) contacting a sample with the dried aliquot of the nitrocefin indicator to form a mixture in the paper; and
c) incubating the mixture;
wherein a beta-lactamase enzyme in a sample comprising the beta-lactamase enzyme is detected by the change in the color of the nitrocefin indicator relative to a control sample when the nitrocefin indicator contacts the beta-lactamase enzyme.

17. The method of claim 16 wherein the concentration of each aliquot of the nitrocefin indicator is about 0.1 mM to about 2 mM.

18. The method of claim 17 wherein the volume of each aliquot of the nitrocefin indicator that is dispersed in the paper within the hydrophobic perimeter is about 1 μL to about 10 μL.

19. The method of claim 16 wherein the nitrocefin indicator comprises a buffer.

20. The method of claim 19 wherein detection of the beta-lactamase enzyme has a limit of detection of about 0.1 mU/mL to about 25 mU/mL.

21. The method of claim 20 wherein the sample comprises blood, blood plasma, or antimicrobial-resistant bacteria.
